(19) United States
(12) Patent Application Publication
(10) Pub. No.: US 2012/0301839 A1
(43) Pub. Date: Nov. 29, 2012

(54) METHOD AND APPARATUS FOR THE ASSESSMENT OF PULPAL VITALITY USING LASER SPECKLE IMAGING

(75) Inventors: Charles Stoianovici, Palm Desert, CA (US); Bernard Choi, Irvine, CA (US); Petra Wilder-Smith, Long Beach, CA (US)

(73) Assignee: The Regents of the University of California, Oakland, CA (US)

(21) Appl. No.: 13/304,275

(22) Filed: Nov. 23, 2011

Related U.S. Application Data
(60) Provisional application No. 61/422,065, filed on Dec. 10, 2010.

(51) Int. Cl. A61B 6/14; A61B 1/24

(52) U.S. Cl. 433/27; 433/224

(57) ABSTRACT

The invention includes a noninvasive method for characterizing dental pulpal vitality of a tooth including the steps of irradiating the tooth with at least partially coherent light, and speckle imaging to provide quantitative feedback to an end user of pulpal blood flow in the tooth, and the tangible records made by such a method. The invention also includes an apparatus for noninvasively characterizing dental pulpal vitality of a tooth comprising a source of at least partially coherent light for irradiating the tooth characterized by a speckle pattern, optics for directing the at least partially coherent light onto the tooth, including a corresponding pulpal cavity within the tooth, a detector for detecting transmission of the at least partially coherent light through the tooth, and a processor coupled to the detector speckle imaging to provide a quantitative feedback of pulpal blood flow in the tooth.

### RELATED APPLICATIONS

The present application is related to U.S. Provisional Patent Application Ser. No. 61/422,065, filed on Dec. 10, 2010, which is incorporated herein by reference and to which priority is claimed pursuant to 35 USC 119.

### GOVERNMENT RIGHTS

This invention was made with government support under contract P41-RR001192 awarded by the National Institutes of Health. The government has certain rights in the invention.

### BACKGROUND

1. Field of the Technology

The disclosure relates to the use of laser speckle imaging in the field of dentistry.

2. Description of the Prior Art

The pulpal chamber of each tooth contains the vasculature necessary to maintain a viable tooth. A critical need exists to develop an objective, repeatable method to assess pulpal viability. Measurement of pulpal vitality is a challenge due to the anatomy of the human mouth and highly scattering nature of teeth. Current methods of measuring blood flow are highly sensitive and user specific making them unreliable.

This problem has not been solved in the past. Previously, the problem was addressed using electrical sensors to determine the vitality of the tooth. The problem with using electrical sensing to determine vitality is that it only determines if the tooth has sensation in the nerves. Vitality can only be determined by a measurement of the blood flow.

The pulpal chamber of each tooth contains the vasculature necessary to maintain a viable tooth. Trauma or infection typically elicit an inflammatory response, which could either resolve on its own or cause the pulpal tissue to become necrotic, leading to gangrene and abscess formation. The most common methods used to assess pulpal health, are hot-and-cold thermal testing and electrical stimulation of the Ad nerve fibers. These methods assess the degree of intact neural innervation in the interrogated tooth. However, recent reports demonstrate that blood flow is a far better indicator of pulpal health. The current inability to accurately diagnose and monitor pulpal perfusion, and its response to noxious stimuli or treatment, provides a strong incentive for clinicians to avoid the risk of attempting measures to maintain pulpal vitality by performing devitalization and root canal therapy as the treatment of choice.

Disadvantages include a long, costly and arduous procedure, medical contraindications, unsuitability of some teeth, as well as long term discoloration and risk of tooth fracture. Research groups have studied the feasibility of various methods to assess the viability of the pulpal chamber.

Others have demonstrated that laser Doppler flowmetry (LDF) can monitor blood flow in the pulpal cavity of intact enamel and dentin. Their data suggest that LDF can differentiate between healthy and necrotic pulpal tissue. However, problems with LDF include a low signal-to-noise ratio and the strong dependence of the signal on the probe position. It has been determined that enamel and dentin are strongly forward scattering and minimally absorb infrared light. This seminal observation led to subsequent investigation of various optical methods such as pulse oximetry and photoplethysmography to assess pulpal vitality. Due to the difficulty in obtaining reproducible data, these methods are not accepted in the dental clinic. Additionally, each method requires the use of a dental splint, further limiting its widespread use.

For widespread clinical acceptance, what is needed is some kind of method ideally involving rapid data collection and not requiring the use of a dental splint.

### BRIEF SUMMARY

The illustrated embodiments of this system are used to assess the vitality of the pulpal chamber of teeth. We hypothesized that the existence of blood perfusion within the pulp can be determined with analysis of laser speckle imaging (LSI) patterns generated by transillumination of the tooth. Laser speckle imaging (LSI) is a noninvasive method of performing wide-field imaging of blood flow. Using LSI, the teeth are transilluminated with light and imaged on the opposite side with a camera. This transillumination approach is used due to the fact that enamel and dentin are materials which scatter light predominantly in the forward direction. A blood flow parameter is quantified from a selected region of interest on the tooth surface.

In the illustrated embodiment our instrument provides real time images of relative blood flow. It acquires images of transilluminated laser light from an object or tooth, and then we employ image processing algorithms to convert the laser transilluminated images, which contain speckle, to speckle-contrast maps and hence relative blood flow maps. Its purpose is to image blood flow dynamics in response to an intervention that is expected to cause blood flow changes. The fundamental principle involves time integrated acquisition of the speckle pattern present in the laser transillumination images. The speckle pattern fluctuates due to motion of scattering particles, such as red blood cells. Instead of analyzing the temporal fluctuations of the speckle pattern, we instead acquire images with relatively long exposure times. By doing this, regions or moving scatterers will appear blurred in the acquired images. The degree of blurring is related to the blood flow and we perform specific image processing steps to quantify the blood flow.

The illustrated embodiments of the method is unique because they provide a semi-quantitative method of measuring blood flow that is minimally invasive and not time-consuming to determine if teeth are vital. The mouthpiece for the system is universal allowing for immediate diagnosis in trauma incidents. In the past, custom mouthpieces needed to be made for other systems to determine vitality which compromised any immediate treatment. An attractive feature of the illustrated embodiments of the invention is that they provide a single blood flow parameter which we believe is a strong indicator of the perfusion within the tooth and hence its vitality.

To address the clinical need, we set out to develop a noninvasive, objective method to characterize pulpal blood flow changes during laser surgery. Laser Doppler flowmetry and laser Doppler imaging, both established methods in studies of microvascular characterization, are limited by the need for mechanical scanning of the probe laser beam, resulting in long image collection times, e.g. on the order of several minutes. In the illustrated embodiments, we instead use laser speckle imaging (LSI) to provide quantitative feedback of pulpal vitality. LSI relies on acquisition and analysis of a single image captured at an exposure time that is considerably longer than a characteristic correlation time associated with the fluctuation frequency. A faster blood flow appears more blurred in the captured image than regions of slower or no flow. The degree of blurring is quantified as the local speckle contrast value, with zero contrast representing no speckle and hence high blood flow, and unity contrast representing a fully developed speckle pattern and hence no flow.

The illustrated embodiments of the invention include a noninvasive method for characterizing dental pulpal vitality of a tooth including the steps of irradiating the tooth with at least partially coherent light, and speckle imaging to provide quantitative feedback to an end user of pulpal blood flow in the tooth.

The step of speckle imaging includes acquiring and analyzing a single image captured at an exposure time that is considerably longer than a characteristic correlation time associated with a fluctuation frequency of the at least partially coherent light.

The step of speckle imaging includes correlating a faster blood flow with a more blurred captured image than in regions of slower or no flow.

The step of correlating a faster blood flow with a more blurred captured image than in regions of slower or no flow includes quantifying the degree of blurring as the local speckle contrast value, with zero contrast representing no speckle and high blood flow, and unity contrast representing a fully developed speckle pattern and no flow.

The illustrated embodiments can also be defined as a noninvasive clinical method for characterizing dental pulpal blood-flow changes in a tooth including the steps of irradiating the tooth with at least partially coherent light, and speckle imaging to provide quantitative feedback to assess the health status of dental pulpal tissue of the tooth and to guide the ensuing course of therapy.

The illustrated embodiments can further be defined as a noninvasive clinical method for characterizing pulpal blood flow in a tooth including the steps of irradiating the tooth with at least partially coherent light; and speckle imaging to provide quantitative feedback by analysis of laser speckle contrast to estimate blood flow changes in the tooth and observed pulpal vitality.

The step of speckle imaging comprises acquiring a time integrated speckle pattern present in a laser transillumination image, wherein the speckle pattern fluctuates due to motion of scattering particles, which image is acquired with relatively long exposure times so that moving scatterers appear blurred in the acquired image and the degree of blurring is related to the blood flow, and image processing to quantify the blood flow.

The illustrated embodiments include an apparatus for noninvasively characterizing dental pulpal vitality of a tooth, which includes a source of at least partially coherent light for irradiating the tooth characterized by a speckle pattern, namely a laser, optics for directing the at least partially coherent light onto the tooth, including a corresponding pulpal cavity within the tooth, a detector or camera for detecting transmission of the at least partially coherent light through the tooth, and a processor or computer coupled to the detector speckle imaging to provide a quantitative feedback of pulpal blood flow in the tooth.

The detector and processor in combination acquire and analyze an image captured at an exposure time that is considerably longer than a characteristic correlation time associated with a fluctuation frequency of the at least partially coherent light.

The processor correlates a faster blood flow with a more blurred captured image than in regions of slower or no flow.

The processor quantifies the degree of blurring as the local speckle contrast value, with zero contrast representing no speckle and high blood flow, and unity contrast representing a fully developed speckle pattern and no flow.

The illustrated embodiments include a tangible record of an image obtained from a noninvasive method for characterizing dental pulpal vitality of a tooth, which record is produced by any one of the methods described above.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIGS. 1a and 1b are schematic diagrams of experimental setups used to evaluate epi-illumination in FIG. 1a and transillumination LSI in FIG. 1b. The buccal and lingual sides of the tooth are those that face the cheek and tongue, respectively.

FIG. 2a illustrates image data fields obtained using transillumination LSI.

FIG. 2b is a graph of the speckle flow index as a function of actual flow speeds for the phantoms of FIG. 2a.

FIG. 3 is a bar graph of the speckle flow index for various flow speeds and angles of incidence for transillumination LSI.

FIG. 4 is a front perspective, diagrammatic view of a prism box illustrated above is employed in an embodiment wherein the illumination laser beam carried in a fiber is coupled via the fiber into a mating hole in a plate fixed to the lower prism.

FIG. 5 is a side cross-sectional view of the prism box of FIG. 4.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

Based on our prior experience with laser speckle imaging (LSI) to study blood-flow dynamics in preclinical animal models and human subjects, we investigated its efficacy in assessing fluid flow in an in vitro tooth model. We hypothesized that the existence of blood perfusion within the pulp can be determined with analysis of laser speckle patterns generated by transillumination of the tooth. This hypothesis is based on our knowledge of the high scattering anisotropy of dental tissue.

We used nine extracted human cuspids and incisors. Upper and lower cuspids and incisors were chosen as they are easy to access in the clinical setting. Samples were stored in water with thymol at a temperature of 0-48° C. We used a diamond-wafering blade (Buehler 10×0.3 mm²) to detach the tooth crown from its root approximately 2-5 mm below the cement-enamel junction (CEJ). The root canal was enlarged to a 1-mm diameter channel from the apical end up to the CEJ using a Rite Dent V1® hand piece equipped with a Whitel HP4® carbide dental bur.

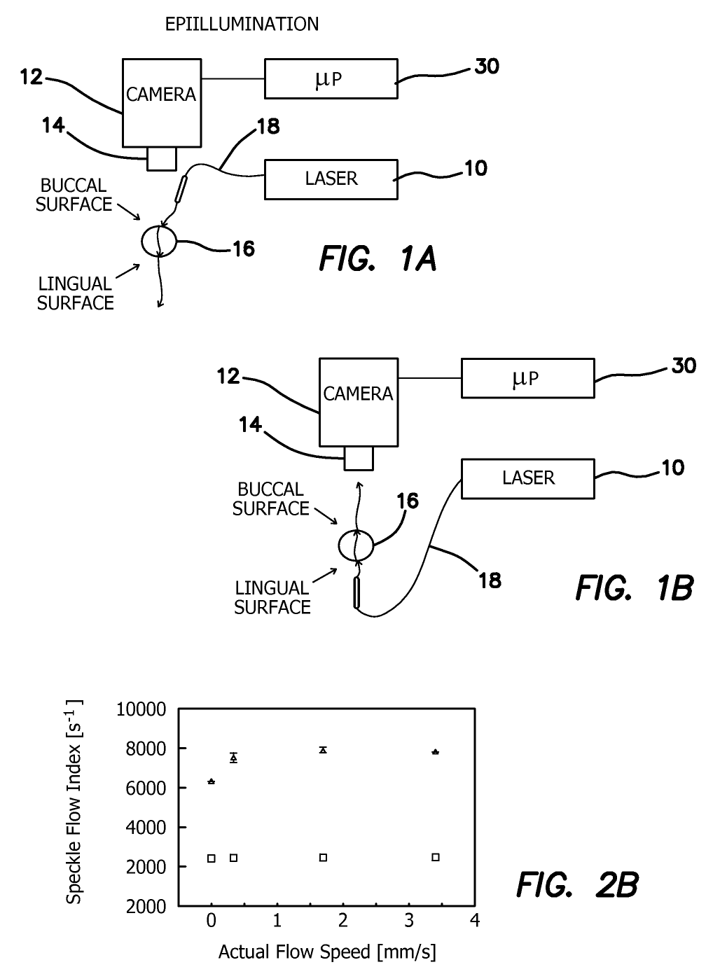

The LSI instrument as shown in two embodiments in FIGS. 1a and 1b was comprised of a laser source 10, a CCD camera 12, and a macro lens 14. Briefly, a 12-bit thermoelectrically cooled CCD camera 12 (1,600×1,200 pixel resolution, Model 2000R®, QImaging, Surrey, Canada) was used to image the raw speckle image remitted from the tooth 16. A 633-nm HeNe laser 10 (30 mW power) was used to transilluminate each tooth sample 16 using an optic fiber 18. By controlling the magnification and aperture of the external macro lens 14, we set the minimum resolvable speckle size to be at least the width of two camera pixels.

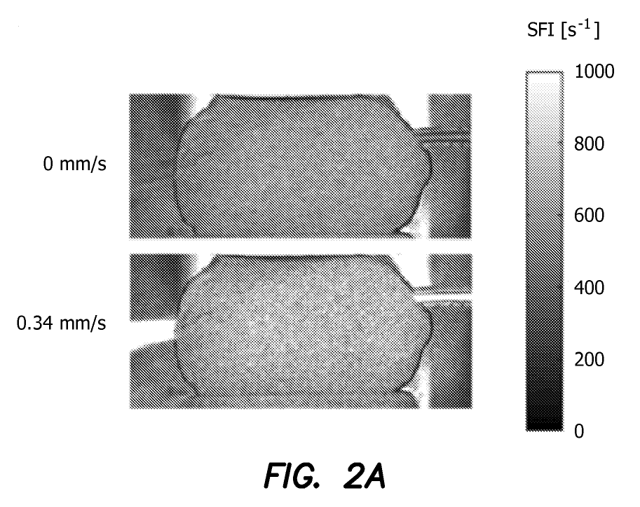

Each raw speckle image first was converted to a speckle contrast image, then to a speckle flow index (SFI) image, such as shown in FIG. 2a, using conventional imaging processing methodology performed in a processor or computer 30 coupled to camera 12. Each set of SFI images was averaged to a single mean SFI image. A region of interest was extracted from each mean SFI image and a single mean SFI value calculated. The selected region included pixels between the top of the root and the CEJ.

To provide a simulative pulpal flow Tygon® tube (not shown) was inserted into a channel created within each tooth 16. The tube had an outer diameter of 1 mm and inner diameter of 0.25 mm. One end of the tube was connected to a 29-gauge insulin syringe. The syringe was mounted onto an infusion pump (Harvard Instruments). The syringe contained 1 ml of a Liposyn® solution. The stock solution (20% intravenous fat emulsion) diluted to a 1:20 Liposyn®:water volume ratio. The tooth 16 was fixed in space with an optomechanical mount. The buccal surface of the tooth 16 was imaged on to the camera 12. We used the following average flow speeds: 0, 0.34, 1.7, and 3.4 mm/second.

We evaluated two imaging configurations. In one set of experiments, we used a transillumination configuration in FIG. 1b, for which laser light was incident on the lingual side (i.e., the side facing the tongue) and resultant speckle pattern imaged from the buccal side (i.e., the side facing the lips). In a second set of experiments, we used an epi-illumination configuration in FIG. 1a, for which laser light was incident on the buccal side and the pattern imaged from the buccal side. For each flow-speed setting and imaging configuration, a sequence of ten raw speckle images was collected. The data were reduced as described above, to a single mean SFI value. Three replicates were performed for each flow-speed setting and imaging configuration. For transillumination experiments, data were collected from nine teeth. For epi-illumination experiments, data were collected from three teeth. For each imaging configuration, a t-test was used to test the null hypothesis that the SFI values collected at 0 mm/second were similar to those collected at 0.34 mm/second (i.e., the lowest nonzero blood-flow speed used)

For in vivo application of LSI, we postulated that the relative angle of incidence of the laser light on the tooth surface, may differ for each tooth 16. To determine the effect of a varying angle of incidence, we performed experiments with the transillumination configuration of FIG. 1b. We used four angles of incidence, relative to normal incidence: 0°, 15°, 30°, and 45°. A 360° manual rotational mount was used to control precisely the angle. For each flow speed setting and relative angle of incidence, a sequence of ten raw speckle images was collected. The data were reduced as described above, to a single mean SFI value. Three replicates were performed for each flow speed setting and angle of incidence. For each flow speed, a single-factor analysis of variance test was used to test the null hypothesis that the SFI values collected at each angle of incidence were similar.

FIG. 2a illustrates measurement with a transillumination LSI configuration with SFI images enabled visualization of a small increase in flow speed. The mean SFI value increased by 70% as the flow speed increased from 0 to 0.34 mm/second. With transillumination LSI (triangles), an increase in SFI values was observed as the fluid flow changed from Brownian fluid motion (0 mm/second) to forced fluid flow (approximately 0.34 mm/second) within the tooth. In contrast, epi-illumination LSI (squares) was insensitive to any change in flow speed. For FIG. 2a, the error bars represent the standard deviation of the mean. For FIG. 2b, the error bars are not visible due to the low coefficient of variation (<3.2%) in measured SFI values.

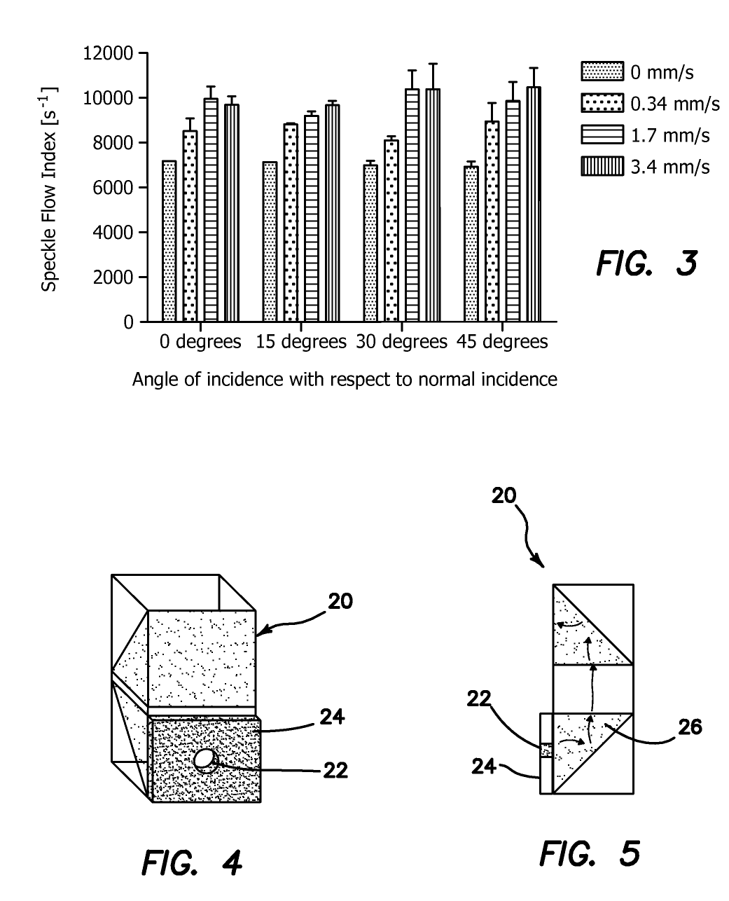

Transillumination LSI, and not epi-illumination LSI, enables differentiation between the absence and presence of perfusion in an in vitro tooth model as illustrated in FIGS. 2a and 2b. With transillumination, an approximate 70% increase in SFI was observed shown in FIG. 2a. In general, the mean SFI values were at least three times higher for transillumination LSI than for epi-illumination LSI, for all flow speeds. For transillumination LSI experiments, the mean SFI value was significantly lower at 0 mm/second than at 0.34 mm/second (P<0.05). For epi-illumination LSI experiments, the mean SFI value was insensitive to flow speed (P>0.30). SFI values are insensitive to the relative angle of incidence of the laser light, over a wide range of angles as illustrated in the graph of FIG. 3. Based on a single-factor analysis of variance test, SFI values for a given flow speed, are identical over a large range of incidence angle settings (P>0.62).

Our preliminary in vitro data support our hypothesis that use of the transillumination LSI method enables determination of the presence of pulpal perfusion. A significant increase in SFI values was observed with a change from stagnant flow to an average flow speed of 0.34 mm/second (FIGS. 2a and 2b). In contrast, epi-illumination LSI was insensitive to flow speeds ranging between 0 and 3.4 mm/second (FIG. 2b).

We propose that transillumination LSI has several advantages over other diagnostic methods which have been described in the peer-reviewed literature. It has the capability to serve as an objective method to study blood flow in the pulpal chamber, as opposed to thermal testing. Furthermore, SFI values obtained with transillumination LSI, were insensitive to the relative angle of incidence of the laser light (FIG. 3). Multiple optical scattering of the light is expected to occur, resulting in a homogenization of both the spatial intensity distribution and the degree to which the speckle pattern is modulated by the moving scatterers in the tube. This result suggests that precise positioning of an eventual probe design in the mouth, is unnecessary to enable accurate interrogation of the pulpal chamber for the presence of blood flow.

Published studies using LDF probes, required the use of stents to affix the probe in place, and the measurements were easily corrupted by relative differences in probe placement or even angulation. To the best of our knowledge, in vivo-measurements of blood flow in the pulp, remain unknown. Arterioles, venules, and capillaries are present in the pulp. If we assume that the blood flow in these vessels is similar to that found in other arterioles, venules, and capillaries in other organs, then the maximum blood flow speed is several mm/second. Hence, the range of flow speeds used in our experimental design, is appropriate to represent in vivo pulpal blood flow. Our data in FIG. 2b suggest that transillumination LSI potentially enables identification of the presence or absence of viable perfusion in the interrogated tooth, but it has limited utility to enable reliable quantization of the actual perfusion value over potential blood-flow speeds in the pulpal vasculature. A possible explanation for this result is the presence of static optical scatterers in the hard dental tissue. Static scattering is known to modulate the measured speckle pattern and diminish the sensitivity of LSI to blood flow.

Additional studies are planned to determine the effects of tooth thickness on measured SFI values and the dynamic range and flow-speed resolution of LSI. Using the advances being made in both laser diode and digital-camera technology, we plan to reduce considerably the footprint of the LSI instrument, to enable development of a probe that could be used with other teeth in addition to incisors. A potential issue is that the intra- and interpatient difference in SFI values in healthy teeth may be greater than the difference observed within our data set. The thickness of the tooth is expected to affect the SFI measurements. To address this issue, various methods can be used to assess tooth thickness, such as the use of calipers. A more difficult issue to address, is that the ratio of dentin to enamel thicknesses is expected to differ for each type of tooth. One approach that we plan to explore in vivo, is to determine the degree of similarity between SFI values associated with contralateral teeth (i.e., the left and right central incisors, the left and right lateral incisors, etc.) from each subject's mouth. If matched teeth are associated with similar SFI values, then transillumination LSI may be used to identify relative differences in blood flow between matched teeth, which may be used as an objective indicator that pulpal blood flow is compromised.

A prism box 20 is illustrated in FIGS. 4 and 5 is employed in an embodiment wherein the illumination laser beam carried in a fiber 18 is coupled via the fiber 18 into a mating hole 22 in a plate 24 fixed to the lower portion of the prism 20. The beam is then redirected 180° and offset by the two prisms 26, 28 and exits from the coupled upper prism 28. The light transmitted light is transmitted through the tooth 16 or more specifically through the pupal chamber of the tooth 16. Camera or detector 12 is then place a few mm's on the opposing side of the tooth 16 and receives the light transmitted through the tooth 16. Use of the prism box 20 is necessitated because of the relative stiffness of the optical fiber 18 and the bulkiness of a conventional camera or detector 12 relative to the available size of the patient's mouth. The fiber 18 cannot be sufficient bent without fracturing to make a acute 180° bend to illuminate the backside of the tooth 16. Only the front facing teeth can be measured by prism box 20 since the camera or detector 12 must be closely spaced to the emitting surface of the tooth 16 and is not yet miniaturized to allow practical insertion within the patient's mouth. It is expected that the apparatus will be miniaturized in additional embodiments where the use of the prism box 20 will not be necessitated or will be sufficiently miniaturized to image any tooth 16 within the patient's mouth.

In conclusion, our preliminary in vitro data suggest that transillumination LSI is a promising method to identify the presence of blood flow in the pulpal chamber. Thus the illustrated embodiments explicitly contemplate a robust in vivo transillumination LSI instrument and pilot clinical data collection to assess intra- and interpatient variability in SFI values.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A noninvasive method for characterizing dental pulpal vitality of a tooth comprising:
irradiating the tooth with at least partially coherent light; and
speckle imaging to provide quantitative feedback to an end user of pulpal blood flow in the tooth.

2. The method of claim 1 where speckle imaging comprises acquiring and analyzing a single image captured at an exposure time that is considerably longer than a characteristic correlation time associated with a fluctuation frequency of the at least partially coherent light.

3. The method of claim 1 where speckle imaging comprises correlating a faster blood flow with a more blurred captured image than in regions of slower or no flow.

4. The method of claim 3 where correlating a faster blood flow with a more blurred captured image than in regions of slower or no flow comprises quantifying the degree of blurring as the local speckle contrast value, with zero contrast representing no speckle and high blood flow, and unity contrast representing a fully developed speckle pattern and no flow.

5. A noninvasive clinical method for characterizing dental pulpal blood-flow changes in a tooth comprising:
irradiating the tooth with at least partially coherent light; and
speckle imaging to provide quantitative feedback to assess the health status of dental pulpal tissue of the tooth and to guide the ensuing course of therapy.

6. A noninvasive clinical method for characterizing pulpal blood flow in a tooth comprising:
irradiating the tooth with at least partially coherent light; and
speckle imaging to provide quantitative feedback by analysis of laser speckle contrast to estimate blood flow changes in the tooth and observed pulpal vitality.

7. The method of claim 6 where speckle imaging comprises acquiring a time integrated speckle pattern present in a laser transillumination image, wherein the speckle pattern fluctuates due to motion of scattering particles, which image is acquired with relatively long exposure times so that moving scatterers appear blurred in the acquired image and the degree of blurring is related to the blood flow, and image processing to quantify the blood flow.

8. An apparatus for noninvasively characterizing dental pulpal vitality of a tooth comprising:
a source of at least partially coherent light for irradiating the tooth characterized by a speckle pattern;
optics for directing the at least partially coherent light onto the tooth, including a corresponding pulpal cavity within the tooth;
a detector for detecting transmission of the at least partially coherent light through the tooth; and
a processor coupled to the detector speckle imaging to provide a quantitative feedback of pulpal blood flow in the tooth.

9. The apparatus of claim 8 where detector and processor in combination acquire and analyze an image captured at an exposure time that is considerably longer than a characteristic correlation time associated with a fluctuation frequency of the at least partially coherent light.

10. The apparatus of claim 8 where the processor correlates a faster blood flow with a more blurred captured image than in regions of slower or no flow.

11. The apparatus of claim 10 where the processor quantifies the degree of blurring as the local speckle contrast value, with zero contrast representing no speckle and high blood flow, and unity contrast representing a fully developed speckle pattern and no flow.

12. A tangible record of an image obtained from a noninvasive method for characterizing dental pulpal vitality of a tooth comprising:
irradiating the tooth with at least partially coherent light; and
speckle imaging to provide quantitative feedback to an end user of pulpal blood flow in the tooth.

13. The tangible record of claim 12 where speckle imaging comprises acquiring and analyzing a single image captured at an exposure time that is considerably longer than a characteristic correlation time associated with a fluctuation frequency of the at least partially coherent light.

14. The tangible record of claim 12 where speckle imaging comprises correlating a faster blood flow with a more blurred captured image than in regions of slower or no flow.

15. The tangible record of claim 14 where correlating a faster blood flow with a more blurred captured image than in regions of slower or no flow comprises quantifying the degree of blurring as the local speckle contrast value, with zero contrast representing no speckle and high blood flow, and unity contrast representing a fully developed speckle pattern and no flow.

16. A tangible record of an image obtained from a noninvasive clinical method for characterizing dental pulpal blood-flow changes in a tooth comprising:
irradiating the tooth with at least partially coherent light; and
speckle imaging to provide quantitative feedback to assess the health status of dental pulpal tissue of the tooth and to guide the ensuing course of therapy.

17. A tangible record of an image obtained from a noninvasive clinical method for characterizing pulpal blood flow in a tooth comprising:
irradiating the tooth with at least partially coherent light; and
speckle imaging to provide quantitative feedback by analysis of laser speckle contrast to estimate blood flow changes in the tooth and observed pulpal vitality.

18. The tangible record of claim 17 where speckle imaging comprises acquiring a time integrated speckle pattern present in a laser transillumination image, wherein the speckle pattern fluctuates due to motion of scattering particles, which image is acquired with relatively long exposure times so that moving scatterers appear blurred in the acquired image and the degree of blurring is related to the blood flow, and image processing to quantify the blood flow.
